SEQUENCE LISTING (8 sequences)

SEQ ID NO: 1 - DNA, 23 bp, synthetic construct
  source 1..23: mol_type = other DNA
tagccttgtcagataaggaagga

SEQ ID NO: 2 - DNA, 20 bp, synthetic construct
  source 1..20: mol_type = other DNA
ctcagcgccatggaaaatgt

SEQ ID NO: 3 - DNA, 26 bp, synthetic construct
  source 1..26: mol_type = other DNA
aaatattgtggatatcgggaggataa

SEQ ID NO: 4 - DNA, 22 bp, synthetic construct
  source 1..22: mol_type = other DNA
aggacggatggaaaaacctgta

SEQ ID NO: 5 - DNA, 21 bp, synthetic construct
  source 1..21: mol_type = other DNA
accacaacatgctgctccttt

SEQ ID NO: 6 - DNA, 22 bp, synthetic construct
  source 1..22: mol_type = other DNA
atcaacgtgcagccatatgtct

SEQ ID NO: 7 - DNA, 22 bp, synthetic construct
  source 1..22: mol_type = other DNA
aacgtcatgacccctcagaagt

SEQ ID NO: 8 - DNA, 21 bp, synthetic construct
  source 1..21: mol_type = other DNA
ttcactgtgcctcaccctgta

(19) United States
(12) Patent Application Publication
(10) Pub. No.: US 2024/0299479 A1
(43) Pub. Date: Sep. 12, 2024

(54) NEPETA CATARIA L. EXTRACT AND METHODS FOR IMPROVING SLEEP AND RESISTING DEPRESSION BY USING THE SAME

(71) Applicant: TCI CO., LTD., Taipei (TW)

(72) Inventors: YUNG-HSIANG LIN, Taipei (TW); FAN-JHEN DAI, Taipei (TW); HUAN-YOU LIN, Taipei (TW)

(21) Appl. No.: 18/422,010

(22) Filed: Jan. 25, 2024

Related U.S. Application Data
(60) Provisional application No. 63/483,304, filed on Feb. 6, 2023.

(30) Foreign Application Priority Data
Dec. 29, 2023 (TW) 112151732

(51) Int. Cl. A61K 36/53; A61K 31/365; A61K 31/7048; A61P 25/20; A61P 39/06

(52) U.S. Cl. CPC A61K 36/53; A61K 31/365; A61K 31/7048; A61P 25/20; A61P 39/06; A61K 2236/331

(57) ABSTRACT

A Nepeta cataria L. extract including one of a compound of formula I and a compound of formula II, or a combination thereof is provided. Methods for shortening sleep latency, improving sleep quality, enhancing vitality of daytime activities, resisting depression, or protecting brain by using the Nepeta cataria L. extract are provided. The Nepeta cataria L. extract is obtained by extracting flower spikes of Nepeta cataria L. with water at 85° C.±5° C.

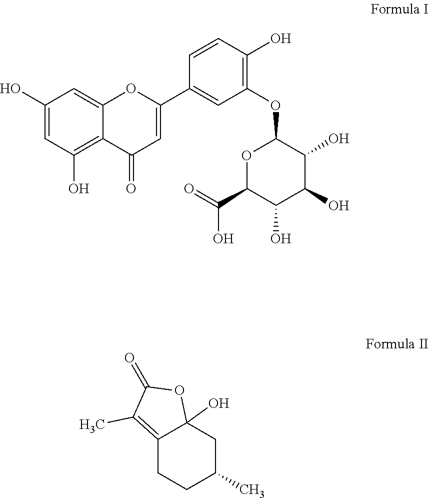

### CROSS-REFERENCES TO RELATED APPLICATIONS

This application claims the benefit of U.S. provisional application Ser. No. 63/483,304, filed on Feb. 6, 2023 and claims the priority of Patent Application No. 112151732 filed in Taiwan, R.O.C. on Dec. 29, 2023. The entirety of the above-mentioned patent applications are hereby incorporated by references herein and made a part of the specification.

### REFERENCE OF ELECTRONIC SEQUENCE LISTING

The contents of the electronic sequence listing (P234176USI.xml; Size: 8,130 bytes; and Date of Creation: Jan. 22, 2024) is herein incorporated by reference in its entirety.

### BACKGROUND

### Technical Field

The present invention relates to a Nepeta cataria L. extract, and particularly relates to a Nepeta cataria L. extract and a method for improving sleep and resisting depression by using the same.

### Related Art

Sleep plays an important role in human health and beauty, allowing the body and skin to be fully rested and restored. There are many causes of insomnia, which may be related to stress, emotions, lifestyle habits, diseases, medication or the like. Insomnia not only harms physical and mental health, but also will increase the risk of suffering from various diseases.

Since the rise of the concept of organic and/or natural diets, biotechnology companies and food businesses have been actively invested in the research and development of products related to natural plants. In order to scientifically verify the benefits of plant-related products to health, the active ingredient analysis and efficacy evaluation of plants have become key projects of product development.

Nepeta cataria L. is a Lamiaceae plant that has a special odor. The biotechnology companies and food businesses are actively conducting the active ingredient analysis and efficacy evaluation of the Nepeta cataria L., and developing related products therefrom.

### SUMMARY

In view of the above, in some embodiments, a use of a Nepeta cataria L. extract for preparing a composition for shortening sleep latency and/or improving sleep quality is provided. The Nepeta cataria L. extract is obtained by extracting flower spikes of Nepeta cataria L. with water at 85° C.±5° C.

In some embodiments, a method for shortening sleep latency and/or improving sleep quality is provided, including administering a composition including a Nepeta cataria L. extract to a subject in need thereof. The Nepeta cataria L. extract is obtained by extracting flower spikes of Nepeta cataria L. with water at 85° C.-5° C.

In some embodiments, the Nepeta cataria L. extract improves melatonin level.

In some embodiments, the Nepeta cataria L. extract reduces a light sleep ratio and/or increases a deep sleep ratio.

In some embodiments, a use of a Nepeta cataria L. extract for preparing a composition for enhancing vitality of daytime activities is provided. The Nepeta cataria L. extract is obtained by extracting flower spikes of Nepeta cataria L. with water at 85° C.±5° C.

In some embodiments, a method for enhancing vitality of daytime activities is provided, including administering a composition including a Nepeta cataria L. extract to a subject in need thereof. The Nepeta cataria L. extract is obtained by extracting flower spikes of Nepeta cataria L. with water at 85° C.±5° C.

In some embodiments, a use of a Nepeta cataria L. extract for preparing a composition for resisting depression is provided. The Nepeta cataria L. extract is obtained by extracting flower spikes of Nepeta cataria L. with water at 85° C.±5° C.

In some embodiments, a method for resisting depression is provided, including administering a composition including a Nepeta cataria L. extract to a subject in need thereof. The Nepeta cataria L. extract is obtained by extracting flower spikes of Nepeta cataria L. with water at 85° C.±5° C.

In some embodiments, the Nepeta cataria L. extract improves serotonin level.

In some embodiments, the Nepeta cataria L. extract reduces neural hyperactivation.

In some embodiments, a use of a Nepeta cataria L. extract for preparing a composition for protecting brain is provided. The Nepeta cataria L. extract is obtained by extracting flower spikes of Nepeta cataria L. with water at 85° C.±5° C.

In some embodiments, a method for protecting brain is provided, including administering a composition including a Nepeta cataria L. extract to a subject in need thereof. The Nepeta cataria L. extract is obtained by extracting flower spikes of Nepeta cataria L. with water at 85° C.±5° C.

In some embodiments, the Nepeta cataria L. extract enhances an antioxidant capability of nerve cells.

In some embodiments, a Nepeta cataria L. extract includes one of a compound of formula I and a compound of formula II, or a combination thereof.

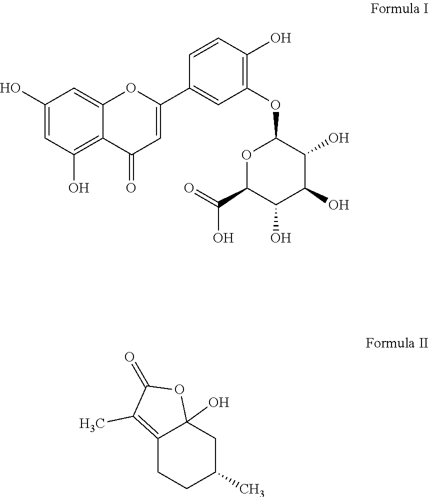

In summary, the Nepeta cataria L. extract of the embodiments of the present disclosure has an effect of improving sleep or resisting depression. In some embodiments, the use of the Nepeta cataria L. extract of the embodiments of the present disclosure for improving sleep or resisting depression relates to a use of a Nepeta cataria L. extract for preparing a composition for improving sleep or resisting depression, thereby providing a composition that can produce an effect of improving sleep or resisting depression on an individual when administered to the individual. In some embodiments, a method for improving sleep or resisting depression is provided, including administering to a subject in need thereof a composition including the Nepeta cataria L. extract of the embodiments of the present disclosure. In other words, the aforementioned composition has the function of improving sleep or resisting depression. In other words, the aforementioned composition can improve sleep of an individual or reduce and/or inhibit depression of an individual after administered to the individual. In some embodiments, the Nepeta cataria L. extract or the composition prepared therefrom also has one or more of the following functions: shortening sleep latency and/or improving sleep quality, enhancing vitality of daytime activities, resisting depression, and protecting brain. In some embodiments, methods for shortening sleep latency, improving sleep quality, enhancing vitality of daytime activities, resisting depression, and protecting brain are provided; the methods includes administering to a subject in need thereof a composition including a Nepeta cataria L. extract.

### BRIEF DESCRIPTION OF THE DRAWINGS

FIG. 1 is a bar chart showing the relative ROS level after treated by a Nepeta cataria L. extract in accordance with some embodiments of the present disclosure.

FIG. 2 is a bar chart showing the relative SIRT1 gene expression level after treated by a Nepeta cataria L. extract in accordance with some embodiments of the present disclosure.

FIG. 3 is a bar chart showing the relative gene expression level after treated by a Nepeta cataria L. extract in accordance with some embodiments of the present disclosure.

FIG. 4 is a bar chart showing the sleep latency in human subjects at week 0 and week 4 after ingesting a composition including a Nepeta cataria L. extract in accordance with some embodiments of the present disclosure.

FIG. 5 is a bar chart showing the relative light sleep ratio at week 0 and week 4 after ingesting a composition including a Nepeta cataria L. extract in accordance with some embodiments of the present disclosure.

FIG. 6 is a bar chart showing the relative deep sleep ratio at week 0 and week 4 after ingesting a composition including a Nepeta cataria L. extract in accordance with some embodiments of the present disclosure.

FIG. 7 is a bar chart showing the sleep quality score at week 0, week 2 and week 4 after ingesting a composition including a Nepeta cataria L. extract in accordance with some embodiments of the present disclosure.

FIG. 8 is a bar chart showing the vitality state score of daytime activities at week 0, week 2, and week 4 after ingesting a composition including a Nepeta cataria L. extract in accordance with some embodiments of the present disclosure.

FIG. 9 is a bar chart showing the serotonin level at week 0, week 2, and week 4 after ingesting a composition including a Nepeta cataria L. extract in accordance with some embodiments of the present disclosure.

FIG. 10 is an HPLC fingerprint spectrum of a Nepeta cataria L. extract in accordance with some embodiments of the present disclosure.

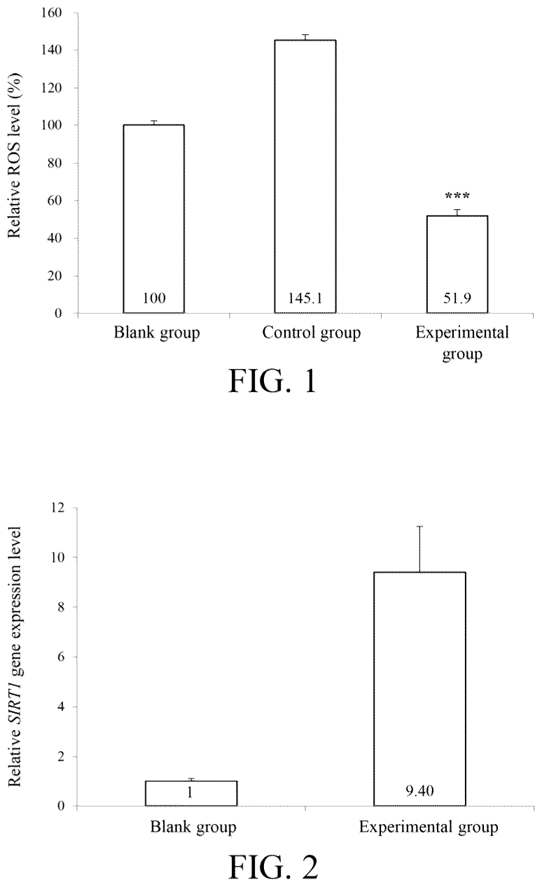

FIG. 1I is a 1H-NMR spectrum of a compound of formula I.

FIG. 12 is a 13C-NMR spectrum of a compound of formula I.

FIG. 13 is a 1H-NMR spectrum of a compound of formula II.

FIG. 14 is a 13C-NMR spectrum of a compound of formula II.

### DETAILED DESCRIPTION

In some embodiments, a Nepeta cataria L. extract is obtained using Nepeta cataria L. as a raw material through an extraction procedure. The extraction procedure mainly includes extracting the Nepeta cataria L. with a solvent to dissolve active ingredients of the Nepeta cataria L. into the solvent.

In some embodiments, the extracted Nepeta cataria L. is flower spikes of the Nepeta cataria L. In some embodiments, the flower spike of the extracted Nepeta cataria L. includes a corolla, a calyx, a receptacle, and a stamen and/or a pistil.

In some embodiments, the extracted Nepeta cataria L. may be intact flower spikes without physical pretreatment (that is, corollas, calyxes, receptacles, and stamens and/or pistils are not separated and their sizes are not physically pretreated), or may be decomposed into fragments, granules, or powder by physical pretreatment. The physical pretreatment used may include at least one of: coarse crushing, chopping, shearing, mashing, and grinding. In some embodiments, the extracted Nepeta cataria L. is intact flower spikes without physical pretreatment.

In some embodiments, the extracted flower spikes may be newly collected flower spikes, dried flower spikes, and/or frozen flower spikes. For example, in the extraction procedure, the dried flower spikes are extracted with a solvent.

In some embodiments, a step of performing the extraction procedure includes extracting Nepeta cataria L. with water at 85° C.±5° C. for 60 min to 90 min to obtain an initial liquid extract. For example, the Nepeta cataria L. can be soaked in the water at 85° C.±5° C. for 60 min to dissolve active ingredients of the Nepeta cataria L. into the water to obtain the initial liquid extract.

In some embodiments, in the extraction procedure, the solvent is water, the raw material is Nepeta cataria L., and the solvent and the raw material have a weight ratio of (10 to 20):1 when initially mixed. For example, the water and the Nepeta cataria L. have a weight ratio of 16:1.

In some embodiments, in the extraction procedure, the initial liquid extract can be further filtered to remove solids such as Nepeta cataria L. extracted with water to obtain a filtrate. For example, the initial liquid extract can be filtered through a 400-mesh filter to remove the solids, and the filtrate is collected.

In some embodiments, in the extraction procedure, the filtrate may be further concentrated to obtain a concentrated solution. In some embodiments, the filtrate can be concentrated under reduced pressure at 55° C. to 65° C. to obtain a concentrated solution. For example, the filtrate can be concentrated under reduced pressure at 60° C.±5° C. In some embodiments, the duration of concentration can be determined by Degrees Brix of the concentrated solution, which is not limited thereto. Following the previous example, the resulting concentrated solution has Degrees Brix of 7.5° Bx±0.5° Bx. In other words, the filtrate can be concentrated under reduced pressure at 60° C.±5° C. until the filtrate obtained after concentration under reduced pressure has 7.5° Bx±0.5° Bx, and the filtrate obtained after the concentration under reduced pressure is the concentrated solution.

In some embodiments, in the extraction procedure, the initial liquid extract can also be concentrated firstly to form a concentrated solution with a reduced liquid volume. Then, the concentrated solution is filtered to remove solids in the concentrated solution, and a filtrate is collected.

In some embodiments, in the extraction procedure, the initial liquid extract is also only concentrated or filtered.

It should be understood that the initial liquid extract, the filtrate, or the concentrated solution obtained by the extraction procedure may be used as the Nepeta cataria L. extract according to actual needs.

In some embodiments, the aforementioned Nepeta cataria L. extract has a capability to shorten sleep latency and/or improve sleep quality. Therefore, the Nepeta cataria L. extract is suitable for preparing a composition for shortening the sleep latency and/or improving the sleep quality.

In some embodiments, a method for shortening sleep latency and/or improving sleep quality is provided, including administering a composition including a Nepeta cataria L. extract to a subject in need thereof.

In some embodiments, the Nepeta cataria L. extract has a capability to improve melatonin level. In other words, when administered to an individual, the Nepeta cataria L. extract can improve melatonin level of the individual. Therefore, the Nepeta cataria L. extract is suitable for preparing a composition for improving melatonin level.

In some embodiments, the Nepeta cataria L. extract has a capability to reduce a light sleep ratio and/or increase a deep sleep ratio. In other words, when administered to an individual, the Nepeta cataria L. extract can reduce a light sleep ratio and/or increase a deep sleep ratio of the individual. Therefore, the Nepeta cataria L. extract is suitable for preparing a composition for reducing a light sleep ratio and/or increasing a deep sleep ratio.

In some embodiments, a method for improving melatonin level, reducing a light sleep ratio, increasing a deep sleep ratio, or any combination thereof is provided, including administering a composition including a Nepeta cataria L. extract to a subject in need thereof.

In some embodiments, the aforementioned Nepeta cataria L. extract has a capability to enhance vitality of daytime activities. Therefore, the Nepeta cataria L. extract is suitable for preparing a composition for enhancing vitality of daytime activities.

In some embodiments, a method for enhancing vitality of daytime activities is provided, including administering a composition including a Nepeta cataria L. extract to a subject in need thereof.

In some embodiments, the aforementioned Nepeta cataria L. extract has a capability to resist depression. Therefore, the Nepeta cataria L. extract is suitable for preparing a composition for resisting depression.

In some embodiments, a method for resisting depression is provided, including administering a composition including a Nepeta cataria L. extract to a subject in need thereof.

In some embodiments, the Nepeta cataria L. extract has a capability to improve serotonin level. In other words, when administered to an individual, the Nepeta cataria L. extract can improve serotonin level of the individual. Therefore, the Nepeta cataria L. extract is suitable for preparing a composition for improving serotonin level.

In some embodiments, the Nepeta cataria L. extract has a capability to reduce neural hyperactivation. In other words, when administered to an individual, the Nepeta cataria L. extract can reduce neural hyperactivation of the individual. Therefore, the Nepeta cataria L. extract is suitable for preparing a composition for reducing neural hyperactivation.

In some embodiments, a method for improving serotonin level, reducing neural hyperactivation, or any combination thereof, including administering a composition including a Nepeta cataria L. extract to a subject in need thereof.

In some embodiments, the aforementioned Nepeta cataria L. extract has a capability to protect brain. Therefore, the Nepeta cataria L. extract is suitable for preparing a composition for protecting brain.

In some embodiments, a method for protecting brain is provided, including administering a composition including a Nepeta cataria L. extract to a subject in need thereof.

In some embodiments, the Nepeta cataria L. extract has a capability to enhance an antioxidant capability of nerve cells. In other words, when administrated to an individual, the Nepeta cataria L. extract can enhance the antioxidant capability of the nerve cells of the individual. Therefore, the Nepeta cataria L. extract is suitable for preparing a composition for enhancing the antioxidant capability of the nerve cells.

In some embodiments, a method for enhancing an antioxidant capability of nerve cells is provided, including administering a composition including a Nepeta cataria L. extract to a subject in need thereof.

In some embodiments, the Nepeta cataria L. extract includes one of a compound of formula I and a compound of formula II, or a combination thereof.

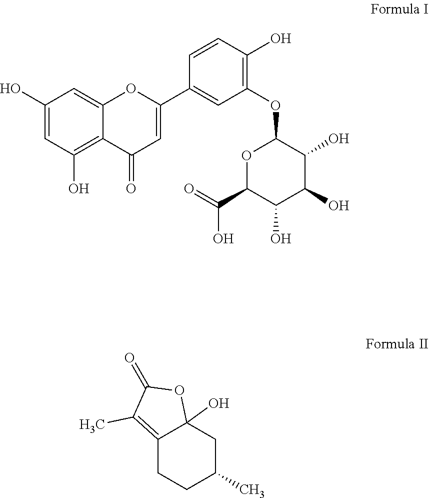

In some embodiments, the compound of formula I is (2S,3S,4S,5R,6S)-6-[5-(5,7-dihydroxy-4-oxo-4H-chromen-2-yl)-2-hydroxyphenoxy]-3,4,5-trihydroxyoxane-2-carboxylic acid. In some embodiments, the compound of formula I is luteolin-3′-glucoronide. The luteolin-3′-glucoronide is a trivial name of the (2S,3S,4S,5R,6S)-6-[5-(5,7-dihydroxy-4-oxo-4H-chromen-2-yl)-2-hydroxyphenoxy]-3,4,5-trihydroxyoxane-2-carboxylic acid.

In some embodiments, the compound of formula II is 7a-hydroxy-3,6-dimethyl-2,4,5,6,7,7a-hexahydrocyclohexa[1,2-b]furan-2-one.

In some embodiments, the aforementioned individual or subject may be a human.

In some embodiments, in the aforementioned composition, the daily intake of the Nepeta cataria L. extract is 3 g.

In some embodiments, the prepared composition may be a pharmaceutical composition or an edible composition for non-medical purposes.

In some embodiments, when the aforementioned composition is a pharmaceutical composition, the pharmaceutical composition includes an effective content of Nepeta cataria L. extract. The pharmaceutical composition can be manufactured into a dosage form suitable for being enterally, parenterally, orally or topically administrated by using a technology known to those skilled in the art.

In some embodiments, the enterally or orally administrated dosage form may be, but is not limited to, a tablet, a troche, a lozenge, a pill, a capsule, dispersible powder, a granule, a solution, a suspension, an emulsion, syrup, an elixir, slurry or the like.

In some embodiments, the parenterally or topically administrated dosage form may be, but is not limited to, an injection [for example, a sterile aqueous solution or dispersion], sterile powder, an external preparation or the like.

In some embodiments, an administration mode of the injection may be, but is not limited to, intraperitoneal injection, subcutaneous injection, intraepidermal injection, intradermal injection, intramuscular injection, intravenous injection or intralesional injection.

In some embodiments, the pharmaceutical composition containing an effective content of Nepeta cataria L. extract may further include a pharmaceutically acceptable carrier that is widely used in a pharmaceutical manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome and the like. The type and quantity of the carrier selected falls within the scope of the professional and routine skills known to those skilled in the art. The solvent used as the pharmaceutically acceptable carrier may be water, normal saline, phosphate buffered saline (PBS) or aqueous solution containing alcohol.

In some embodiments, the pharmaceutical composition containing an effective content of Nepeta cataria L. extract can be manufactured into an external preparation suitable for being topically administrated to skin by using a technology well known to those skilled in this art. In some embodiments, the external preparation includes, but is not limited to: an emulsion, gel, ointment, cream, a patch, liniment, powder aerosol, spray, lotion, serum, paste, foam, a drop, a suspension, salve and a bandage.

In some embodiments, when the aforementioned pharmaceutical composition is the external preparation, the pharmaceutical composition can be made by mixing an effective content of Nepeta cataria L. extract with a base known to those skilled in the art.

In some embodiments, the base may include one or more additives selected from the following additives: water, alcohols, glycol, hydrocarbons [such as petroleum, jelly and white petroleum], wax [such as paraffin and yellow wax], preserving agents, antioxidants, surfactants, absorption enhancers, stabilizing agents, gelling agents [such as Carbopol®974P, microcrystalline cellulose and carboxymethylcellulose], active agents, humectants, odor absorbers, fragrances, pH adjusting agents, chelating agents, emulsifiers, occlusive agents, emollients, thickeners, solubilizing agents, penetration enhancers, anti-irritants, colorants, propellants and the like. The selection and quantity of these additives falls within the scope of the professional and routine skills known to those skilled in the art.

In some embodiments, when the aforementioned composition is an edible composition for non-medical purposes, the edible composition includes an effective content of Nepeta cataria L. extract. The edible composition may be in a form of powder, granules, solutions, colloid or paste.

In some embodiments, the edible composition containing the Nepeta cataria L. extract may be a food product or a food additive.

In some embodiments, the edible composition containing the Nepeta cataria L. extract may be beverages, fermented foods, bakery products, health foods, dietary supplements or the like. In some embodiments, the edible composition containing the Nepeta cataria L. extract may further includes an adjuvant. For example, the adjuvant may be maltodextrin, malic acid, sucralose, citric acid, a fruit flavor, a honey flavor, steviol glycoside or a combination thereof. The type and quantity of the adjuvant selected falls within the scope of the professional and routine skills known to those skilled in the art.

In some embodiments, the food additive may be a seasoning, a sweetener, a flavor, a pH adjusting agent, an emulsifier, a colorant a stabilizer or the like.

Unless otherwise specified in the following examples, the experimental steps are carried out at room temperature (25° C. to 30° C.) and atmospheric pressure (1 atm).

### Example 1: Preparation of Nepeta Cataria L. Extract

### A. Materials:

1. Flower spikes of Nepeta Cataria L. (origin: China).
2. Secondary water, also referred to as RO (reverse osmosis) water or secondary distilled water, hereinafter referred to as “water”.

### B. Preparation Flow:

1. The water was heated to 85° C.±5° C., then the flower spikes of the Nepeta Cataria L. were added, and were soaked in water at 85° C.±5° C. for 60 min to form an initial liquid extract containing solids. Here, the water and the added Nepeta cataria L. had a weight ratio of 16:1.
2. The cooled initial liquid extract was filtered through a 400-mesh filter to remove the solids (i.e., the flower spikes of the extracted Nepeta cataria L.) to obtain a filtrate.
3. A temperature of a concentrator (model: Rotavapor R-100; brand: BUCHI) was set as 60° C.±5° C., and the filtrate was concentrated under reduced pressure by using the concentrator. When the filtrate was concentrated until degrees Brix was 7.5° Bx±0.5° Bx, the concentration was stopped to obtain a concentrated solution.
4. The concentrated solution was filtered through a 400-mesh filter to obtain a Nepeta Cataria L. extract.

### Example 2: Antioxidation Test of Nerve Cell

### A. Materials and Instruments:

1. Cell line: Mouse brain neuroblastoma cell, purchased from ATCC (American Type Culture Collection), cell number: CCL-131™, hereinafter referred to as a Neuro2a cell.
2. Cell medium: DMEM (Dulbecco's Modified Eagle's medium) (purchased from Gibco, product number: 12100-046), supplemented with 10% fetal bovine serum (purchased from Gibco, product number: 10437-028), 1% antibiotic-antimycotic (purchased from Thermo, product number: 15240062), and 3.7 g/L sodium bicarbonate (purchased from Sigma, product number: S8875-5006).
3. Trypsin: Prepared from 10× trypsin (purchased from Gibco, product number: 15400-054) and 9 times the volume of DPBS by dilution.
4. 35% H₂O₂ solution: prepared from H₂O₂ (purchased from Sigma, product number: 1.08600) and PBS.
5. DCFH-DA treatment reagent: prepared from DCFH-DA (2′,7′-dichlorodihydrofluorescein diacetate) (purchased from Sigma, product number: SI-D6883-50MG) and DMSO (purchased from Sigma, product number: 472301).
6. Flow cytometer, model: BD Accuri, purchased from BD.

### B. Test Flow:

1. The Neuro2a cells were inoculated into 6-well culture plate containing 2 mL of cell medium per well at a density of 2×10⁵ Neuro2a cells per well, and were cultured at 37° C. for 24 h. Here, the Neuro2a cells were divided into three test groups: a blank group, a control group and an experimental group, respectively. Each group was repeated two times.
2. After 24 h of culture, the cell medium in each group was replaced with an experimental medium. The experimental medium in the blank group and the experimental medium in the control group were each a cell medium without a sample, and the experimental medium in the experimental group was a cell medium containing 0.25% (v/v) of Nepeta cataria L. extract prepared in Example 1. Next, each group was subjected to treatment at 37° C. for 1 h.
3. After 1 h of the treatment, the DCFH-DA treatment reagent was added to each group until its final concentration was 5 μg/mL, and each group was subjected to treatment at 37° C. for 15 min.
4. After 15 min of the treatment, 35% H₂O₂ solution was added to the control group and the experimental group until its final concentration was 1 mM, and each group was subjected to treatment at 37° C. for 1 h.
5. After treatment, the experimental medium in each group was removed, and each group was rinsed twice with PBS.
6. After the rinsing, 200 μL trypsin was added into each well for a reaction in the dark for 5 min. After the reaction, the cell medium was added to stop the reaction. Then suspended cells and the cell medium in each well were collected into a corresponding centrifuge tube, and each centrifuge tube was centrifuged to precipitate the cells.
7. Supernatant in the centrifuge tube in each group was removed, and the precipitated cells were washed with the PBS once. Then 1 mL PBS was added into the centrifuge tube in each group to resuspend the cells to form a cell suspension.
8. A parameter of excitation light of the flow cytometer was set as 450 to 490 nm, and a parameter of emission light of the flow cytometer was set as 510 to 550 nm. Then a green fluorescence signal of each group was detected by using the flow cytometer.

### C. Test Results:

A relative ROS (reactive oxygen species) level of each group was calculated according to the following formula: relative ROS level (%)=(green fluorescence signal value of each group/green fluorescence signal value of blank group)×100%.

Statistically significant differences between test results of the control group and other groups were statistically analyzed by a student t-test. In the figure, “*” represents that the p values are less than 0.05 when compared with the control group, “**” represents that the p values are less than 0.01 when compared with the control group, and “***” represents that the p values are less than 0.001 when compared with the control group.

Refer to FIG. 1. The cells in the blank group were neither treated with a sample nor irritated with H₂O₂. Therefore, the test results of the blank group represent expression of the cells under normal physiological metabolism condition. Here, in a case where the relative ROS level of the blank group was set as 100%, the relative ROS level of the control group was 145.1%, while the relative ROS level of the experimental group was 51.9%. That is, compared to that of the blank group, the relative ROS level of the control group was increased by about 45.1% after the cells in the control group were irritated with H₂O₂. Compared to that of the control group, the relative ROS level of the experimental group was significantly reduced by about 64.2% after the cells in the experimental group were treated with the Nepeta Cataria L. extract and were irritated with H₂O₂. Compared to that of the blank group, the relative ROS level of the experimental group was reduced by about 48.1%.

From this, it can be learned that the Nepeta Cataria L. extract can significantly reduce the ROS level of nerve cells caused by H₂O₂. H₂O₂ can induce the cells to undergo aerobic metabolism, thus producing ROS. ROS within cells can attack large molecules such as proteins, nucleic acids, and lipids, causing cell damage. In other words, it has been experimentally proven that the Nepeta Cataria L. extract has a capability to enhance the antioxidant capability of the nerve cells and reduce nerve cell damage. The Nepeta Cataria L. extract has an effect of enhancing antioxidant activity and reducing cell damage.

### Example 3: Gene Test of Nerve Cell

### A. Materials and Instruments:

1. Cell Line: Human neuroblastoma cell, purchased from ATCC, cell number: CRL-2266™, hereinafter referred to as an SHSY-5Y cell.
2. Cell medium: DMEM (Dulbecco's Modified Eagle's medium) (purchased from Gibco, product number: 11965-092), supplemented with 10% fetal bovine serum (purchased from Gibco, product number: 10437-028) and 1% antibiotic-antimycotic (purchased from Gibco, product number: 15240-062).
3. RNA extraction kit, purchased from TAN Bead, product number: 301538.
4. SuperScript® III reverse transcriptase, purchased from Invitrogen, product number: 18080-051.
5. ABI StepOnePlus™ Real-Time PCR system, purchased from Thermo Fisher Scientific.
6. KAPA SYBR FAST qPCR Master Mix (2×) Kit, purchased from Sigma, product number: KM4102.

### B. Test Flow:

1. The SHSY-5Y cells were inoculated into 6-well culture plate containing 2 mL of cell medium per well at a density of 1×10⁶ SHSY-5Y cells per well, and were cultured at 37° C. for 24 h. Here, the SHSY-5Y cells were divided into two test groups: a blank group and an experimental group, respectively. Each group was repeated three times.
2. After 24 h of culture, the cell medium in each group was replaced with an experimental medium. The experimental medium in the blank group was a cell medium without a sample, and the experimental medium in the experimental group was a cell medium containing 0.25% (v/v) of Nepeta cataria L. extract prepared in Example 1. Next, the cells in each group were cultured at 37° C. for 24 h.
3. After 24 h of culture, the SHSY-5Y cells in each group were collected. Next, RNA in the SHSY-5Y cells in each group was extracted with the RNA extraction kit.
4. 1000 nanograms (ng) of the extracted RNA in each group was used as a template, and the extracted RNA was reverse-transcribed with the SuperScript® III reverse transcriptase into corresponding cDNA.
5. A quantitative real-time reverse transcription polymerase chain reaction was carried out on the cDNA in each group by using the ABI StepOnePlus™ Real-Time PCR system, with the KAPA SYBR FAST qPCR Master Mix (2×) Kit and primers pairs in Table 1 to observe an expression level of each target gene of the SHSY-5Y cells in the blank group and the experimental group and melting curve thereof. Instrument set conditions for the quantitative real-time reverse transcription polymerase chain reaction were as follows: reaction at 95° C. for 20 s, reaction at 95° C. for 3 s, and reaction at 60° C. for 30 s, which was repeated for 40 cycles.
6. A relative expression level of the target gene was determined by using a 2^{-ΔΔCt} method. The so-called relative expression level is defined as a fold change of an RNA expression level of the target gene in the experimental group or the blank group compared to that of the same gene in the blank group. Here, the quantitative real-time reverse transcription polymerase chain reaction by the cDNA can indirectly quantify an mRNA expression level of a gene, thereby inferring an expression level of a protein coded by the gene. In the 2^{-ΔΔCt} method, using a cycle threshold of a GAPDH (glyceraldehyde 3-phosphate dehydrogenase) gene as a cycle threshold (Ct) of a reference gene for internal control, the fold change was calculated using the following formula:

ΔCt=Ct_{target gene of experimental group/target gene of blank group}−Ct_{GAPDH}

ΔΔCt=ΔCt_{target gene of experimental group}−ΔCt_{target gene of blank group}

Fold change=2⁻ΔΔ^{Ct mean}

TABLE 1

| Target | Primer | Sequence | |
|---|---|---|---|
| gene | name | number | Sequence |
| SIRT1 | SIRT1-F | SEQ ID | TAGCCTTGTCAGATAAGGAAGGA |
| | | NO: 1 | |
| | SIRT1-R | SEQ ID | CTCAGCGCCATGGAAAATGT |
| | | NO: 2 | |
| TPH1 | TPH1-F | SEQ ID | AAATATTGTGGATATCGGGAGGA |
| | | NO: 3 | TAA |
| | TPH1-R | SEQ ID | AGGACGGATGGAAAAACCTGTA |
| | | NO: 4 | |
| DDC | DDC-F | SEQ ID | ACCACAACATGCTGCTCCTTT |
| | | NO: 5 | |
| | DDC-R | SEQ ID | ATCAACGTGCAGCCATATGTCT |
| | | NO: 6 | |
| AANAT | AANAT-F | SEQ ID | AACGTCATGACCCCTCAGAAGT |
| | | NO: 7 | |
| | AANAT-R | SEQ ID | TTCACTGTGCCTCACCCTGTA |
| | | NO: 8 | |

### C. Test Results:

A relative target gene expression level of each group was calculated according to the following formula: relative target gene expression level=(target gene expression level of each group/target gene expression level of blank group).

Statistically significant differences between test results of the blank group and the experimental group were statistically analyzed by a student t-test. In the figure, “*” represents that the p values are less than 0.05 when compared with the blank group, “**” represents that the p values are less than 0.01 when compared with the blank group, and “***” represents that the p values are less than 0.001 when compared with the blank group.

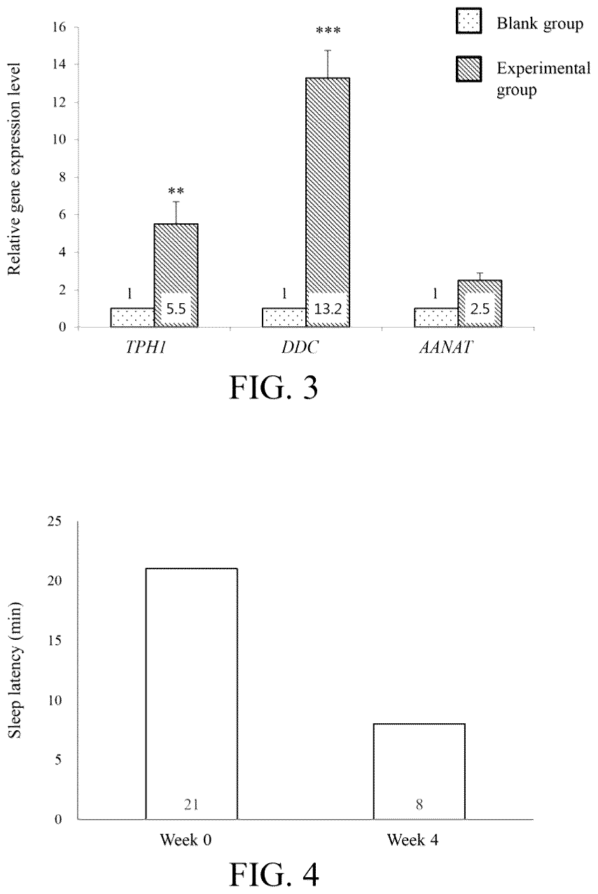

Refer to FIG. 2. The cells in the blank group were not treated with a sample. Therefore, the test results of the blank group represent expression of the cells under normal physiological metabolism condition. Here, in a case where the relative SIRT1 gene expression level of the blank group was set as 1 time, the relative SIRT1 gene expression level of the experimental group was 9.40 times. That is, compared to that of the blank group, the relative SIRT1 gene expression level of the experimental group was increased by about 840% after the cells in the experimental group were treated with the Nepeta cataria L. extract.

From this, it can be learned that the Nepeta cataria L. extract can increase the expression level of an SRI gene of nerve cells. The SIRT1 gene is responsible for producing Sirt1 (Sirtuin 1), where Sirt1 has been shown to be associated with depression, and the expression of Sirt1 in a patient with the depression is significantly lower than that in a healthy subject. Sirt1 inhibits hyperactivation of brain glial cells to prevent the occurrence of depressive emotional states, and can also inhibit emotional disorders caused by inflammation of nerve cells, and can improve emotions. In other words, it has been experimentally proven that the Nepeta cataria L. extract has the effect of reducing and/or inhibiting hyperactivation of the nerve cells, reducing the depression, and also has the effect of reducing and/or inhibiting inflammations of the nerve cells, reducing the emotional disorders, and improving the emotions. The Nepeta cataria L. extract has an effect of resisting depression and resisting depressive disorders.

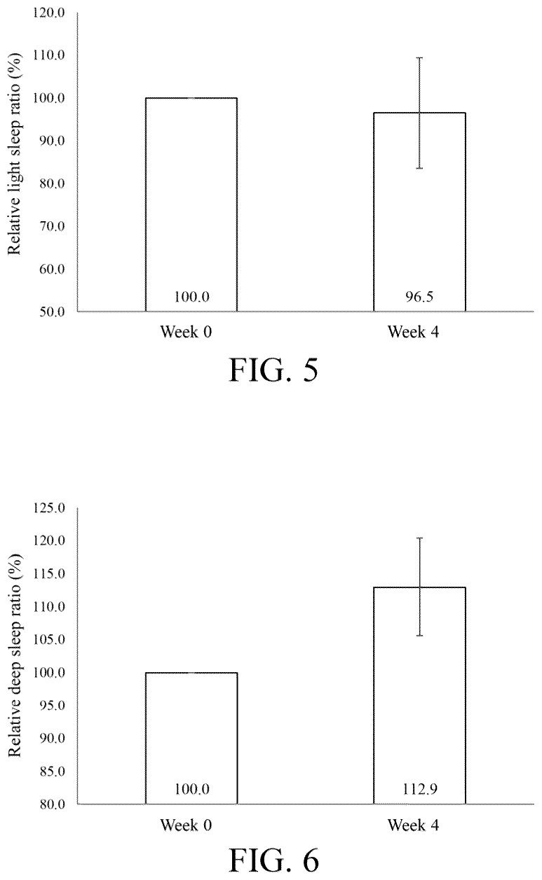

Refer to FIG. 3. The cells in the blank group were not treated with a sample. Therefore, the test results of the blank group represent expression of the cells under normal physiological metabolism condition. Here, in a case where the relative TPH1 gene expression level, the relative DDC gene expression level and the relative AANAT gene expression level of the blank group were set as 1 time, the relative TPH1 gene expression level of the experimental group was 5.5 times, the relative DDC gene expression level of the experimental group was 13.2 times, and the relative AANAT gene expression of the experimental group was 2.5 times. That is, compared to that of the blank group, the relative TPH1 gene expression level of the experimental group was significantly increased by about 450% after the cells in the experimental group were treated with the Nepeta cataria L. extract. Compared to that of the blank group, the relative DDC gene expression level of the experimental group was significantly increased by about 1220% after the cells in the experimental group were treated with the Nepeta cataria L. extract. Compared to that of the blank group, the relative AANAT gene expression level of the experimental group was significantly increased by about 150% after the cells in the experimental group were treated with the Nepeta cataria L. extract.

From this, it can be learned that the Nepeta cataria L. extract can increase the expression levels of TPH1 and DDC genes of the nerve cells. The TPH1 gene is responsible for producing tryptophan hydroxylase 1 (TPH1), where TPH1 is responsible for synthesizing serotonin. The DDC gene is responsible for producing an enzyme to be involved in production of dopamine and serotonin. In other words, it has been experimentally proven that the Nepeta Cataria L. extract has an effect of enhancing synthesis of the serotonin by the nerve cells. The Nepeta cataria L. extract has an effect of improve the serotonin level. Since the serotonin can keep people awake and happy, the Nepeta cataria L. extract has an effect of keeping people awake and happy.

From this, it can be learned that the Nepeta cataria L. extract can increase the expression level of an AANAT gene of the nerve cells. The AANAT gene is a key regulatory gene for the circadian rhythm of a human body, and is responsible for producing an enzyme to be involved in synthesis of melatonin, where the enzyme is an important enzyme that catalyzes conversion of the serotonin into the melatonin. In other words, it has been experimentally proven that the Nepeta Cataria L. extract has an effect of increasing conversion of the serotonin into the melatonin in the nerve cells and the synthesis of the melatonin in the nerve cells. The Nepeta cataria L. extract has an effect of improve the melatonin level. Since the melatonin can make people sleep peacefully and regulate the operation of a biological clock, the Nepeta cataria L. extract has an effect of helping people sleep at night, making people sleep peacefully and regulating the biological clock.

### Example 4: Human Subject Test

### A. Test Flow:

7 adult subjects aged 20 to 55 with poor mental condition or poor sleep status were instructed to take one bottle of test drink daily for 4 consecutive weeks (i.e., 28 days). The test drink contained 3 g of Nepeta cataria L. extract prepared in Example 1 and 47 g of water. Moreover, the subjects underwent a sleep test, a somatosensory questionnaire survey, and a blood test before the start of taking (hereinafter referred to as week 0), 14 days after taking (hereinafter referred to as week 2), and 28 days after taking (hereinafter referred to as week 4).

An electrocardiograph was used in the sleep test to test a light sleep ratio, a deep sleep ratio, and sleep latency of the subjects before and after taking. Based on a CPC heart-lung coupling theory developed by Harvard University of the USA, the electrocardiograph analyzes a sleep architecture through a heart rate, detects soundly sleep, light sleep, rapid eye movement period sleep, wakefulness and other states per minute of the subject, and accurately monitors sleep data. In the human subject test, the subjects were tested by wearing an electrocardiograph from Largan Health AI-Technology Co., Ltd., Taiwan.

A sleep status questionnaire was used in the somatosensory questionnaire survey to make the subjects to conduct self-assessment before and after taking. The sleep status questionnaire is as shown in Table 2 below. The subjects conducted self-assessment on their sleep quality and vitality states of daytime activities. All the subjects self-assessed their sleep statuses, judged these sleep statuses, and selected one of the following five options: very good (5 points), not bad (4 points), average (3 points), poor (2 points), and very poor (1 point).

TABLE 2

| | | | | |
|---|---|---|---|---|
| Very good | Not bad | Average | Poor | Very poor |
| (5 | (4 | (3 | (2 | (1 |
| points) | points) | points) | points) | point) |

Sleep quality in the
past 2 weeks
Vitality statuses of
daytime activities in
the past 2 weeks

In the blood test, the blood of the subjects under uneaten state were drawn, and serum specimens of the subjects were entrusted to Lezen Clinical Laboratory (Taiwan) to determine changes of the serotonin level in the blood of the subjects before and after taking.

### B. Test Results:

Statistically significant differences between test results at week 0 and other weeks and test results at week 2 and other weeks were statistically analyzed by a student t-test. In the figure, “*” represents that the p values are less than 0.05 when compared with week 0, “**” represents that the p values are less than 0.01 when compared with week 0, and “***” represents that the p values are less than 0.001 when compared with week 0. In the figure, “#” represents that the p values are less than 0.05 when compared with week 2, “##” represents that the p values are less than 0.01 when compared with week 2, and “###” represents that the p values are less than 0.001 when compared with week 2.

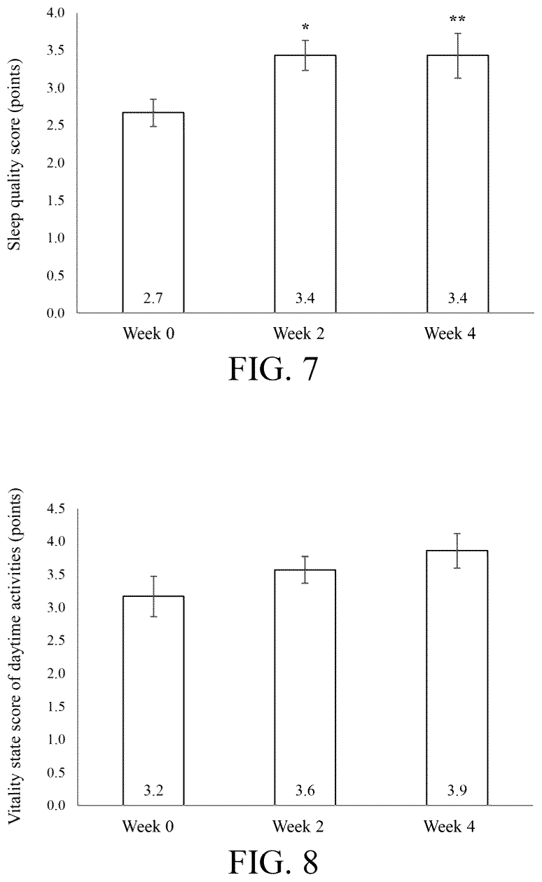

Refer to FIG. 4. FIG. 4 shows the sleep latency of one of the subjects before and after taking the test drink. The sleep latency of the subject at week 0 was 21 minutes, while the sleep latency of the subject at week 4 (i.e. after taking the Nepeta cataria L. extract for 4 consecutive weeks) was 8 minutes. In other words, compared to before taking, the sleep latency of the subject can be shortened by 13 minutes, i.e., 61.9%, after taking the Nepeta cataria L. extract for 4 consecutive weeks. From this, it can be learned that the Nepeta cataria L. extract can indeed shorten the sleep latency. In other words, the Nepeta cataria L. extract has an effect of helping to fall asleep quickly and stay asleep.

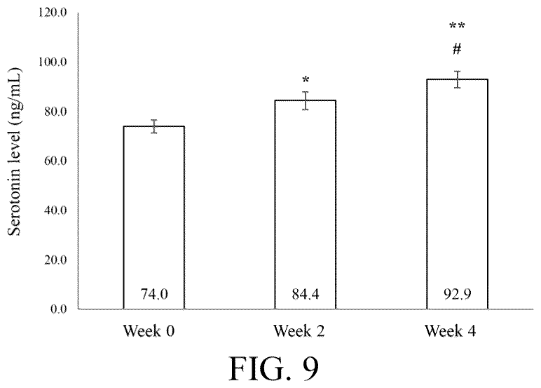

Refer to FIG. 5. FIG. 5 shows an average relative light sleep ratio of the 7 subjects before and after taking the test drink. A light sleep ratio of the 7 subjects measured before taking was considered as 100% of relative light sleep ratio. At this time, the relative light sleep ratio at week 4 (i.e., after taking the Nepeta cataria L. extract for 4 consecutive weeks) was 96.5%. In other words, compared to before taking, the relative light sleep ratio of these subjects can be reduced by 3.5% after taking the Nepeta cataria L. extract for 4 consecutive weeks. From this, it can be learned that the Nepeta cataria L. extract can indeed reduce the light sleep ratio.

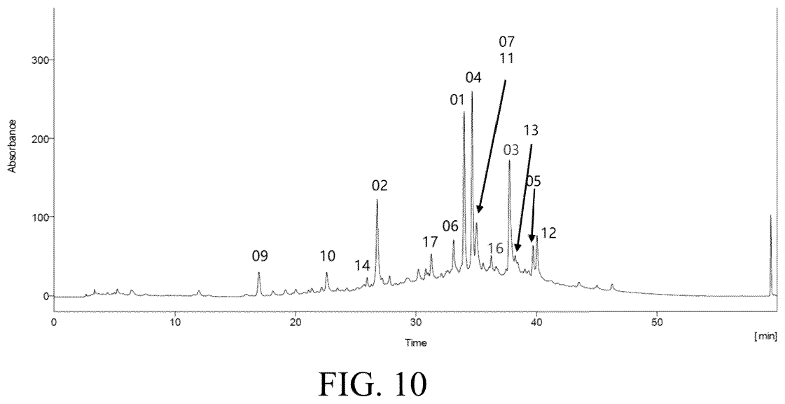

Refer to FIG. 6. FIG. 6 shows an average relative deep sleep ratio of the 7 subjects before and after taking the test drink. A deep sleep ratio of the 7 subjects measured before taking was considered as 100% of relative deep sleep ratio. At this time, the relative deep sleep ratio at week 4 (i.e., after taking the Nepeta cataria L. extract for 4 consecutive weeks) was 112.9%. In other words, compared to before taking, the relative deep sleep ratio of these subjects can be increased by 12.9% after taking the Nepeta cataria L. extract for 4 consecutive weeks. From this, it can be learned that the Nepeta cataria L. extract can indeed increase the deep sleep ratio. In other words, the Nepeta cataria L. extract has an effect of increasing deep sleep. Since the brain and body are in a state of complete rest in a deep sleep state, which is main sleep to eliminate physical fatigue, the Nepeta cataria L. extract has an effect of allowing the body to rest completely and relieving fatigue.

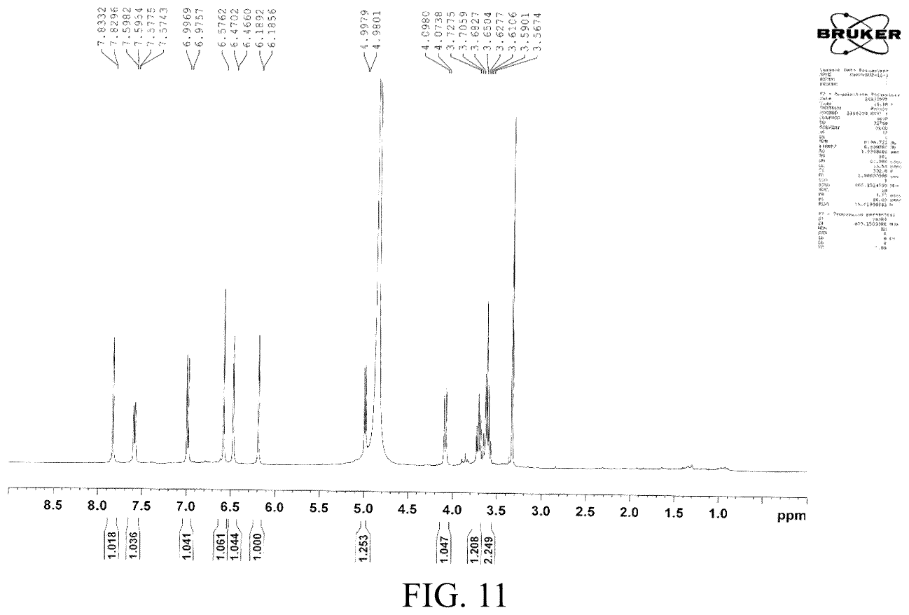

Refer to FIG. 7. FIG. 7 shows an average sleep quality score of the 7 subjects before and after taking the test drink. The sleep quality score at week 0 was 2.7 points, the sleep quality score at week 2 (i.e., after taking the Nepeta cataria L. extract for 2 consecutive weeks) was 3.4 points, and the sleep quality score at week 4 (i.e., after taking the Nepeta cataria L. extract for 4 consecutive weeks) was 3.4 points. In other words, compared to before taking, the sleep quality score of these subjects can be significantly increased by 25.9% after taking the Nepeta cataria L. extract for 2 consecutive weeks, while the sleep quality score of these subjects can be significantly increased by 25.9% after taking the Nepeta cataria L. extract for 4 consecutive weeks. Moreover, a percentage of individual improved at week 2 was 57.1% (4 individuals), while a percentage of individual improved at week 4 was 85.7% (6 individuals). From this, it can be learned that the Nepeta cataria L. extract can indeed improve the sleep quality.

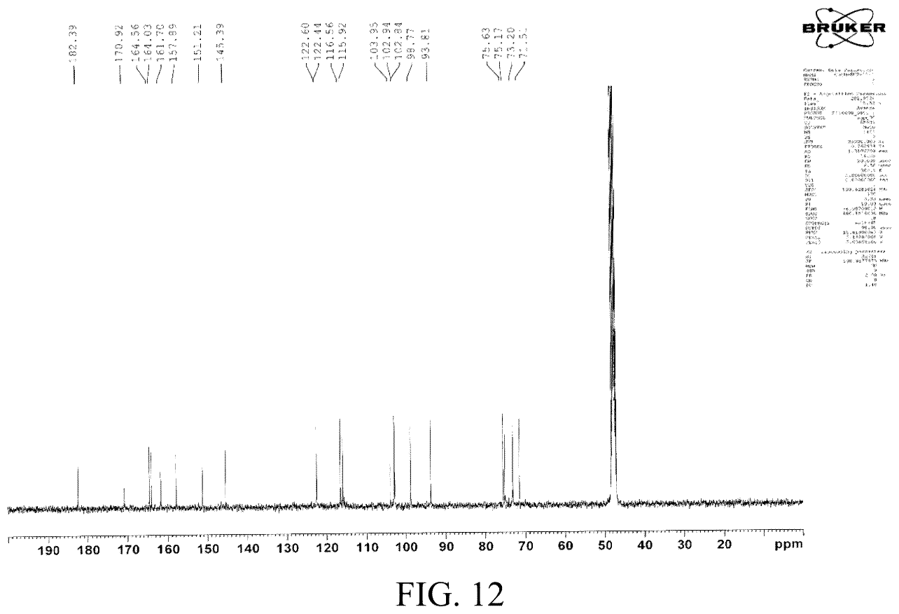

Refer to FIG. 8. FIG. 8 shows an average vitality state score of daytime activities of the 7 subjects before and after taking the test drink. The vitality state score of daytime activities at week 0 was 3.2 points, the vitality state score of daytime activities at week 2 (i.e., after taking the Nepeta cataria L. extract for 2 consecutive weeks) was 3.6 points, and the vitality state score of daytime activities at week 4 (i.e., after taking the Nepeta cataria L. extract for 4 consecutive weeks) was 3.9 points. In other words, compared to before taking, the vitality state score of daytime activities of these subjects can be increased by 12.5% after taking the Nepeta cataria L. extract for 2 consecutive weeks, while the vitality state score of daytime activities of these subjects can be increased by 21.9% after taking the Nepeta cataria L. extract for 4 consecutive weeks. Moreover, the percentage of individuals improved was 85.7% (6 individuals). From this, it can be learned that the Nepeta cataria L. extract can indeed enhance the vitality of daytime activities. In other words, the Nepeta cataria L. extract has an effect of enhancing daytime vitality and daytime mental status.

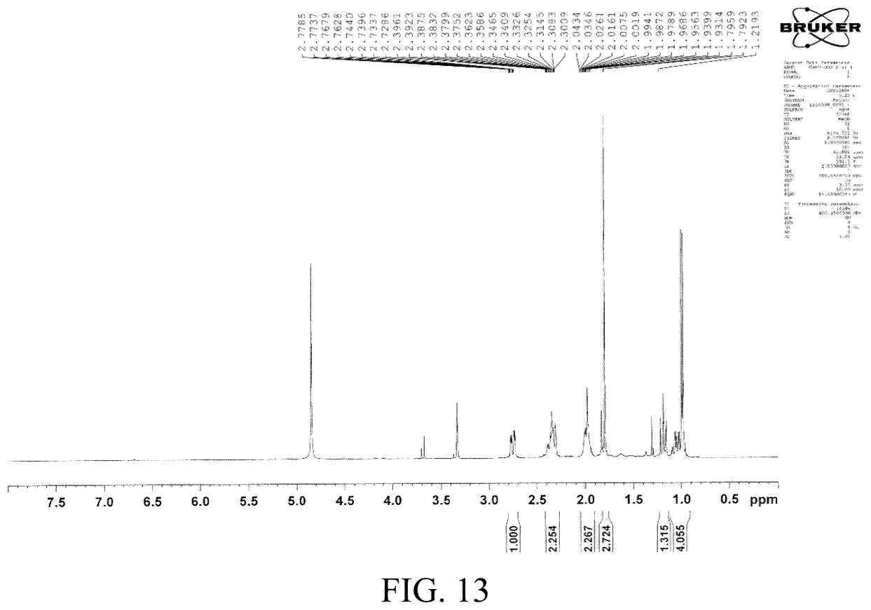

Refer to FIG. 9. FIG. 9 shows average serotonin level of the 7 subjects before and after taking the test drink. The serotonin level at week 0 was 74.0 ng/mL, the serotonin level at week 2 (i.e., after taking the Nepeta cataria L. extract for 2 consecutive weeks) was 84.4 ng/mL, and the serotonin level at week 4 (i.e., after taking the Nepeta cataria L. extract for 4 consecutive weeks) was 92.9 ng/mL. In other words, compared to before taking, the serotonin level of these subjects can be significantly increased by 14.1% after taking the Nepeta cataria L. extract for 2 consecutive weeks, while the serotonin level of these subjects can be significantly increased by 25.5% after taking the Nepeta cataria L. extract for 4 consecutive weeks. Compared to after taking for 2 weeks, the serotonin level of these subjects can be significantly increased by 10.1% after taking the Nepeta cataria L. extract for 4 consecutive weeks. Moreover, a percentage of individual improved at week 2 was 85.7% (6 individuals), while a percentage of individual improved at week 4 was 100% (7 individuals). From this, it can be learned that the Nepeta cataria L. extract can indeed increase the serotonin level. Serotonin is one of raw materials of melatonin. If there is a lack of serotonin, it is more likely to cause insomnia or sleep disorders. In other words, the Nepeta cataria L. extract can indeed improve sleep quality.

### Example 5: Compound Analysis Test

### A. Materials and Instruments:

1. Nuclear magnetic resonance spectrometer (NMR): Ascend 400 MHz was used for 1D and 2D spectra, purchased from Bruker Co., Germany, and chemical shift was represented by 6 in ppm.
2. High resolution liquid chromatography mass spectrometer: Tandem ultra performance liquid chromatography (Ultimate 3000 HPLC, purchased from Thermo Fisher Scientific) and high-resolution orbitrap mass spectrometer (Q-EXACTIVE System with Ion Max Source, purchased from Thermo Fisher Scientific) for determination in m/z.
3. Medium pressure liquid chromatography (MPLC): CombiFlash® Rf+, purchased from Teledyne ISCO, Lincoln, Nebraska, USA.
4. High performance liquid chromatography (HPLC): Hitachi chromaster 5260 series, purchased from Hitachi, Tokyo, Japan; an elution solvent delivery pump was Hitachi chromaster 5110; a column thermostatic device was Hitachi chromaster 5310; and a diode array detector (DAD) was Hitachi chromaster 5430, with a detection wavelength of 250 nm.
5. HPLC analytical column: Mightysil RP-18 GP 250 (250×4.6 mm, 5 μm, purchased from Kanto, Tokyo, Japan).
6. HPLC fractionation column: Luna@ 5 μm C18 (2) 100 Å (250×10 mm), purchased from Phenomenex, USA.
7. Column chromatography filling materials:
(1) Macroporous resin: Diaion HP-20, purchased from Mitsubishi Chemical Company, Japan.
(2) Positive phase silicone: Merck Kieselgel 60, 40-63 um, purchased from Merck, Germany, product number: Art. 9385.
(3) Reverse phase silicone: Merck LiChroprep® RP-18, 40-63 um, purchased from Merck, Germany, product number: Art. 0250.
8. Thin-Layer Chromatography:
(1) TLC aluminum sheet: Thin-layer chromatography sheet, coated with silicone 60 F254 (0.25 mm), purchased from Merck, Germany.
(2) TLC aluminum sheet: Thin-layer chromatography sheet, coated with RP-18 F254-S (0.25 mm), purchased from Merck, Germany.
9. UV lamp: UVP UVGL-25 with wavelengths of 254 nm and 365 nm.
10. Solvents: Methanol, acetonitrile, n-butanol, chloroform-d1 (degree of deuteration: 99.5%), methanol-d4 (degree of deuteration: 99.5%), deuterium oxide (degree of deuteration: >99.8%), and dimethyl sulfoxide-d6 (degree of deuteration: >99.9%), all purchased from Merck, Taiwan.
11. Ultra-pure water (18.2 M□): Taken from Millipore Synergy® water preparation system (purchased from Millipore, USA).
12. Chemical reagents: Methanol (HPLC grade) and formic acid (ACS grade), purchased from Merck, Taiwan.
13. Filter membrane: Polyvinylidene fluoride membrane filters (PVDFs) with a pore size of 0.22 microns, purchased from Millipore, USA.
14. Sample: The Nepeta cataria L. extract prepared in Example 1.

### B. HPLC Analysis Flow:

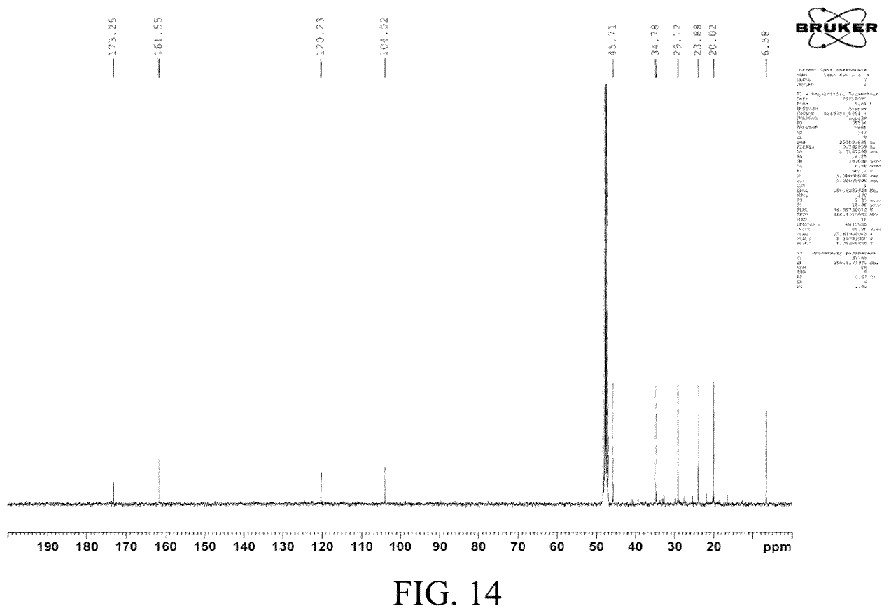

1. The Nepeta cataria L. extract was filtered using the filter membrane.
2. The Nepeta cataria L. liquid extract was analyzed by using HPLC. Here, methanol (A) and water (B) were used as solvents, 0.1% formic acid was added, and a flow rate was set as 1 mL/min and the injection amount of the Nepeta cataria L. extract was set as 10 μL. Elution conditions: methanol (A):water (B)=2:98 at 0 min; A:B=2:98 at 10 min; A:B=70:30 at 40 min; A:B=100:0 at 50 min; and A:B=100:0 at 60 min. During HPLC analysis, the column temperature was 40° C.
3. Refer to FIG. 10. An HPLC fingerprint spectrum of the Nepeta cataria L. extract exhibits numerous distinct peaks (including peaks 01-07, 09-14, and 16-17).

### C. Compound Fractionation and Structural Identification Flow:

1. In order to fractionate a liposoluble ingredient and a water-soluble ingredient of the Nepeta cataria L. extract, 10 L of the Nepeta cataria L. extract was subjected to liquid-liquid phase partition extraction using n-butanol as the solvent to obtain 42.3 g of n-butanol soluble fraction (liposoluble ingredient) and 131.3 g of water-soluble fraction (water-soluble ingredient).
2. During the fractionation and purification process of a functional compound, bioassay guided fractionation was used.
3. The 42.3 g of n-butanol soluble fraction was preliminarily fractionated further with Diaion HP-20 macroporous resin column chromatography fractionation, and 3 fractionated fractions (hereinafter referred to as a BUF1 fractionated fraction, a BUF2 fractionated fraction and a BUF3 fractionated fraction) were obtained by using pure water, pure water/methanol at a volume ratio of 1:1, and methanol as an elution solution sequentially.
4. The BUF-3 fractionated fraction was taken and re-fractionated using a reverse-medium pressure liquid chromatograph (RP-MPLC) to obtain multiple eluates. Here, linear elution was performed with the elution solution from the water to the methanol for 120 min at a flow rate of 10 ml/min. Subsequently, similar eluates were merged using the thin-layer chromatography (TLC aluminum sheet: thin-layer chromatography sheet, coated with RP-18 F254-S (0.25 mm)) to obtain 5 sub-fractionated fractions (hereinafter referred to as a BUF3-1 fractionated fraction, a BUF3-2 fractionated fraction, a BUF3-3 fractionated fraction, a BUF3-4 fractionated fraction, and a BUF3-5 fractionated fraction).
5. The BUF3-3 fractionated fraction was purified via reverse-HPLC (methanol/water=11/9) to obtain compound TCI-CatM-03. After a chemical structure of the compound TCI-CatM-03 was analyzed by 1H-NMR and 13C-NMR, by data comparison with Reference 1 (Flavonoid and phenolic glycosides from Salvia offcinalis. Yinrong Lu, L. Yeap Foo. Phytochemistry, 2000, 55, 263-267.), the compound TCI-CatM-03 was confirmed to be (2S,3S,4S,5R,6S)-6-[5-(5,7-dihydroxy-4-oxo-4H-chromen-2-yl)-2-hydroxyphenoxy]-3,4,5-trihydroxyoxane-2-carboxylic acid, English name: luteolin-3′-glucoronide, as shown in FIG. 11 to FIG. 12.
6. The BUF3-2 fractionated fraction was purified via reverse-HPLC (methanol/water=1/1) to obtain compound TCI-CatM-16. After a chemical structure of the compound TCI-CatM-16 was analyzed by 1H-NMR and 13C-NMR, by data comparison with Reference 2 (Synthesis and Characterization of Bimetallic Nanoclusters Stabilized by Chiral and Achiral Polyvinylpyrrolidinones. Catalytic C(sp3)-H Oxidation. Huafang Fan, Zongbo Tong, Zhaoyang Ren, Kanchan Mishra, Shunya Morita, Edruce Edouarzin, Lingaraju Gorla, Boris Averkiev, Victor W. Day, Duy H. Hua. J. Org. Chem. 2022, 87, 10, 6742-6759.), the compound TCI-CatM-16 was confirmed to be 7a-hydroxy-3,6-dimethyl-2,4,5,6,7,7a-hexahydrocyclohexa[1,2-b]furan-2-one, as shown in FIG. 13 to FIG. 14.

The names and chemical structural formulas of the aforementioned compounds are shown in Table 3.

TABLE 3

| Compound | Name | Chemical structural formula |
|---|---|---|
| TCI-CatM-03 | (2S,3S,4S,5R,6S)-6-[5-(5,7- dihydroxy-4-oxo-4H-chromen- 2-yl)-2-hydroxyphenoxy]-3,4,5- trihydroxyoxane-2-carboxylic acid | 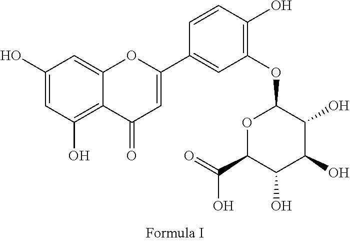 [SMILES: O=C(O)[C@H]1O[C@@H](Oc2cc(-c3cc(=O)c4c(O)cc(O)cc4o3)ccc2O)[C@H](O)[C@@H](O)[C@@H]1O] |
| TCI-CatM-16 | 7a-hydroxy-3,6-dimethyl- 2,4,5,6,7,7a- hexahydrocyclohexa[1,2- b]furan-2-one | 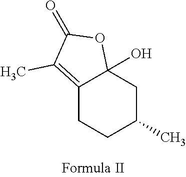 |

In summary, the Nepeta cataria L. extract of the embodiments of the present disclosure has an effect of improving sleep or resisting depression. In some embodiments, the use of the Nepeta cataria L. extract of the embodiments of the present disclosure for improving sleep or resisting depression relates to a use of a Nepeta cataria L. extract for preparing a composition for improving sleep or resisting depression, thereby providing a composition that can produce an effect of improving sleep or resisting depression on an individual when administered to the individual. In some embodiments, a method for improving sleep or resisting depression is provided, including administering to a subject in need thereof a composition including the Nepeta cataria L. extract of the embodiments of the present disclosure. In other words, the aforementioned composition has the function of improving sleep or resisting depression. In other words, the aforementioned composition can improve sleep of an individual or reduce and/or inhibit depression of an individual after administered to the individual. In some embodiments, the Nepeta cataria L. extract or the composition prepared therefrom also has one or more of the following functions: shortening sleep latency and/or improving sleep quality, enhancing vitality of daytime activities, resisting depression, and protecting brain. In some embodiments, methods for shortening sleep latency, improving sleep quality, enhancing vitality of daytime activities, resisting depression, and protecting brain are provided; the methods includes administering to a subject in need thereof a composition including a Nepeta cataria L. extract.

What is claimed is:

1. A method for shortening sleep latency and/or improving sleep quality, comprising administering to a subject in need thereof a composition comprising a Nepeta cataria L. extract, wherein the Nepeta cataria L. extract is obtained by extracting flower spikes of Nepeta cataria L. with water at 85° C.±5° C.

2. The method according to claim 1, wherein the Nepeta cataria L. extract improves melatonin level of the subject.

3. The method according to claim 1, wherein the Nepeta cataria L. extract reduces a light sleep ratio and/or increases a deep sleep ratio of the subject.

4. A method for enhancing vitality of daytime activities, comprising administering to a subject in need thereof a composition comprising a Nepeta cataria L. extract, wherein the Nepeta cataria L. extract is obtained by extracting flower spikes of Nepeta cataria L. with water at 85° C.±5° C.

5. A method for resisting depression, comprising administering to a subject in need thereof a composition comprising a Nepeta cataria L. extract, wherein the Nepeta cataria L. extract is obtained by extracting flower spikes of Nepeta cataria L. with water at 85° C.±5° C.

6. The method according to claim 5, wherein the Nepeta cataria L. extract improves serotonin level of the subject.

7. The method according to claim 5, wherein the Nepeta cataria L. extract reduces neural hyperactivation of the subject.

8. A method for protecting brain, comprising administering to a subject in need thereof a composition comprising a Nepeta cataria L. extract, wherein the Nepeta cataria L. extract is obtained by extracting flower spikes of Nepeta cataria L. with water at 85° C.±5° C.

9. The method according to claim 8, wherein the Nepeta cataria L. extract enhances antioxidant capability of nerve cells of the subject.

10. A Nepeta cataria L. extract, comprising one of a compound of formula I and a compound of formula II, or a combination thereof. [chemical structure]
